(19) United States
(12) Patent Application Publication
(10) Pub. No.: US 2012/0148654 A1
(43) Pub. Date: Jun. 14, 2012

(54) METHOD AND SYSTEM FOR A PHOTORESIST-BASED IMMUNOISOLATIVE MICROCONTAINER WITH NANOSLOTS DEFINED BY NANOIMPRINT LITHOGRAPHY

(76) Inventors: Barjor Gimi, Jamaica Plain, MA (US); Wenchuang Hu, Allen, TX (US); Jeong-Bong Lee, Plano, TX (US)

(21) Appl. No.: 12/966,179

(22) Filed: Dec. 13, 2010

(51) Int. Cl. A61F 2/00; A61K 38/22; A61K 38/18; C12N 11/00; A61P 3/10; A61P 25/28; A61P 35/00; A61K 35/12; A61P 5/00

(52) U.S. Cl. 424/422; 424/93.7; 514/9.7; 514/7.6; 435/174

(57) ABSTRACT

The present technology provides a system and/or method for a photoresist-based immunoisolative microcontainer with nanoslots defined by nanoimprint technology. The present technology further provides a method of immunoisolating one or more biomolecules, the method comprising providing a biocompatible microcontainer, wherein the microcontainer comprises a base and semi-permeable nanoporous surface. Methods of using the microcontainers for transplantation and cell therapy are also described.

### FEDERALLY SPONSORED RESEARCH OR DEVELOPMENT

This invention was made with government support under Grant No. 1R01EB007456, awarded by the National Institutes of Health. The government has certain rights in this invention.

### CROSS-REFERENCE TO RELATED APPLICATIONS/INCORPORATION BY REFERENCE

[Not Applicable]

### FIELD OF THE INVENTION

The present technology relates generally to the field of biotherapeutics, including cell therapy and transplantation. The present technology provides immunoprotective, nanoporous microcontainers for encapsulating biomolecules and/or cells. In some embodiments, these microcontainers may be transplanted into an individual to provide therapeutic benefits, such as secretion of certain proteins and/or molecules without being recognized by the immune system. Certain embodiments of the invention relate to biotherapeutic molecule transplantation. More specifically, certain embodiments of the invention relate to a method and system for a photoresist-based immunoisolative microcontainer with nanoslots defined by nanoimprint technology.

### BACKGROUND OF THE INVENTION

A number of diseases can be treated by biotherapeutics, including cell therapy and/or cell transplantation. One major problem in cell therapy and/or cell transplantation is the rejection of the donor graft or cells by the recipient's immune system and thus the need for immunosuppressive drugs to reduce or eliminate the immune reaction to the foreign cells. For some diseases, such as hormone deficiencies, for example, diabetes (which is caused by the lack of insulin production by the pancreas), treatment includes hormone replacement via hormone injections. Other approaches have been to transplant cells producing the hormone into a patient, such as pancreas transplantation or islet transplantation. But transplantation of organs is limited by a short supply of donor organs and the immune system's response to the donor organ or cells. Right now, transplant recipients must be placed on immunosuppressive drugs to inhibit the recipient's immune system from attacking and destroying the donated tissue or cells. Numerous postoperative complications in organ transplantation also can occur including rejection and/or side effects associated with the long term use of immunosuppressive drugs. There is a need in the art for new methods of providing immunoprotection of the grafted organ and/or cells, and the site specific delivery of cells that can produce certain proteins and/or biomolecules.

Prior to the present technology, microcapsules made of alginate hydrogel (a marine polysaccharide) were one of the most common approaches in cell encapsulation therapy. However, alginate-based microcapsules have exhibited a broad distribution of pore sizes, which in turn allows undesired immune components to diffuse through the microcapsule and eventually leads to the destruction of encapsulated cells. Alginate-based microcapsules also exhibit insufficient resistance to organic solvents and inadequate mechanical strength. Microelectromechanical systems (MEMS)-based biocapsules have provided uniform membrane porosity and mechanical and chemical stability. However, these biocapsules are on the order of several millimeters and therefore are not small enough to be implanted in many sites. Self-folded cubic containers have also been studied for generic microassembly application and cell encapsulation applications but also has disadvantages in that these devices are primarily made of non-biocompatible materials and are fabricated using high temperatures and harmful chemicals.

There is a need in the art for biocompatible microcontainers for use in cell transplantation and cell therapy which providing immunoprotection from the host's immune system, especially microcontainers that have well controlled and uniform nanoporosity.

### BRIEF SUMMARY OF THE INVENTION

In one aspect, the present technology provides a system and/or method for a photoresist-based immunoisolative microcontainer with nanoslots defined by nanoimprint technology, substantially as shown in and/or described in connection with at least one of the figures, as set forth more completely in the claims.

In another aspect, the present technology provides a method of immunoisolating one or more biomolecules, the method comprising providing a biocompatible photoresist-based microcontainer, wherein the photoresist-based microcontainer comprises a base and semi-permeable nanoporous lid. The nanoporous lid includes a thin nanoporous photoresist membrane comprising a dense array of uniform slits and a thick photoresist support layer including a uniform array of cylindrical wells. The method further includes encapsulating the one or more cells within the biocompatible photoresist-based microcontainer.

In another aspect, the present technology provides a method of treating a disease in a patient, comprising administering one or more cells which have been immunoisolated in the microcontainer of the present technology. The diseases which may be treated include hormone deficiency diseases, diabetes, parathyroid disease, Parkinson's disease, hemophilia, Alzheimer's disease, CNS malignancies, hypothyroidism, and cancer among others.

In yet a further embodiment, the present technology provides a method of treating a disease or disorder in a patient comprising administering to the patient one or more cells encapsulated within an immunoisolating microcontainer, wherein the immunoisolating microcontainer comprises a cuboid base and semi-permeable nanoporous lid, wherein the nanoporous lid comprising a thin nanoporous photoresist membrane comprising a dense array of slits and a thick photoresist support layer comprising an array of cylindrical wells; and wherein the microcontainer comprises a biocompatible and non-immunogenic composition.

Various advantages, aspects and novel features of the present invention, as well as details of an illustrated embodiment thereof, will be more fully understood from the following description and drawings.

### BRIEF DESCRIPTION OF SEVERAL VIEWS OF THE DRAWINGS

FIG. 1 is a diagram illustrating an exemplary photoresist-based microcontainer, in accordance with an embodiment of the invention.

FIG. 2 is a diagram illustrating an exemplary process for fabricating a photoresist-based microcontainer, in accordance with an embodiment of the invention.

FIG. 3 is a diagram illustrating an exemplary process for fabricating a silicon mold for nanoimprinting, in accordance with an embodiment of the invention.

FIG. 4 illustrates top views from a scanning electron microscope of a silicon grating, in accordance with an embodiment of the invention.

FIG. 5 is a diagram illustrating an exemplary process for fabricating a nanoporous lid for a microcontainer, in accordance with an embodiment of the invention.

FIG. 6 shows a scanning electron microscope image of the cross section of an imprinted and etched photoresist membrane, in accordance with an embodiment of the invention.

FIG. 7 shows scanning electron microscope images of a microcontainer with nanoporous lid, in accordance with an embodiment of the invention.

### DETAILED DESCRIPTION OF THE INVENTION

The present technology relates generally to the field of biotherapeutics, including cell therapy and transplantation. In some embodiments of the present technology provide an immunoprotective, cell-encapsulating nanoporous microcontainer. In other embodiments, microcontainer containing one or more biomolecules are provided that can be transplanted into an individual to provide therapeutic benefits. For example, microcontainers comprising cells may be used to secrete certain proteins and/or molecules without being recognized by the immune system. Encapsulated cell therapy can alter or restore endogenous function for many cell types and diseases.

Certain aspects of the present technology may be found in a method and system for a photoresist-based immunoisolative microcontainer with nanoslots defined by nanoimprint technology. The microcontainers of the present technology have controlled and uniform porosity within their lid structures, unlike tortuous polymers. This controlled porosity effectively prevents the passage of immune complement compounds through the surface of microcontainers. The porosity of the present technology provides precise and reproducible pore sizes, allowing only certain molecules to pass through the membrane. The microcontainer size and structure makes it mechanically stable and robust, unlike biocapsules with principally two-dimensional thin membranes that can rupture. Unlike self-assembling microcontainers which spontaneously fold and therefore cannot be loaded after assembly, the microcontainers of the present technology have an open surface that permits loading after partial assembly, upon which time they can be closed without the use of harmful chemicals or high temperatures. The present technology uses novel methods to create nanopores using electron beam lithography and nanoimprinting result in extremely precise control over dimensions. Methods for high-throughput biofriendly fabrication are useful for translational and commercially viable applications.

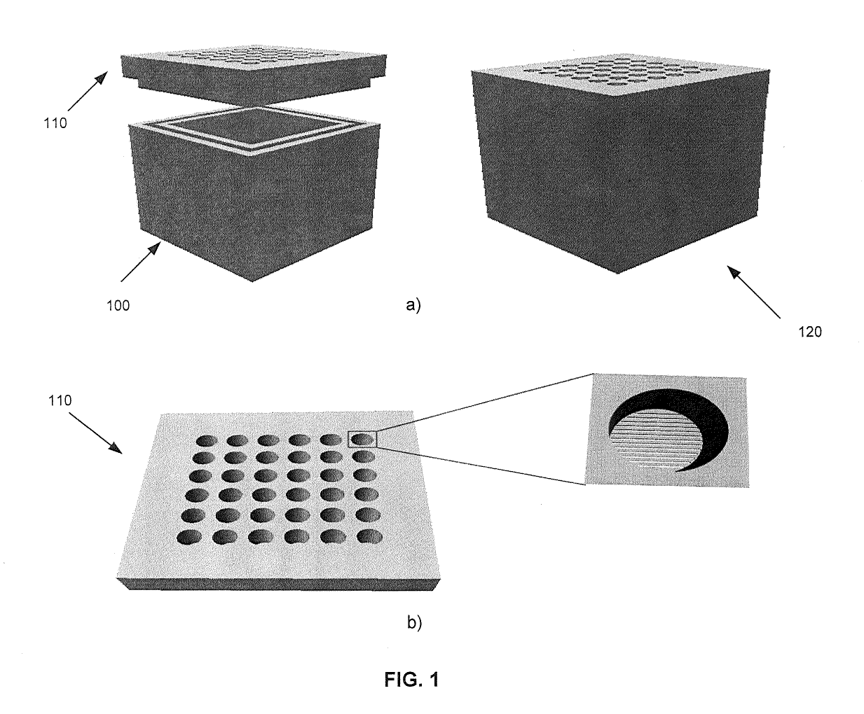

FIG. 1 is a diagram illustrating an exemplary photoresist-based microcontainer, in accordance with an embodiment of the present technology. Referring to FIG. 1, there is shown a cuboid base 100, a nanoporous lid 110, and an assembled microcontainer 120. The microcontainer 120 may be utilized for the treatment of diseases or disorders, for example, by transplantation of cells into a patient in need thereof. For example, the microcontainer 120 can be used for treatment of diseases, including, but not limited to, hormone deficiency diseases, diabetes, parathyroid disease, Parkinson's disease, hemophilia, Alzheimer's disease, CNS malignancies, hypothyroidism, diseases that can be cured by biotherapeutic or biotoxic molecules secreted by cells. Further examples of uses of the present technology include stem cell therapy for regeneration and other cell based therapy including immunization and anti-cancer therapy including the secretion/release of anti-cancer molecules.

The microcontainers of the present technology are used to encapsulate or enclose one or more biomolecules or one or more cells and protect them from interactions with the external environment, in some embodiments, the immune system of a transplant recipient. Encapsulation provides a mechanism for the protection of transplanted cells from a host immune system and can eliminate the requirement of immunosuppressive drugs. In one embodiment, the present technology provides an epoxy-polymer-based, or a highly cross-linked polymer based microcontainer with nanoporous surfaces, which provides a means for cell encapsulation. The nanopores allow nutrients to enter the microcontainer and useful biotherapeutic molecules that are secreted by the encapsulated cells to exit the microcontainer. However, the pores do not permit the passage of large molecules of the immune system that can destroy the encapsulated cellular grafts, i.e. the pores serve as a selective molecular filter. The microcontainer provides an avenue to circumvent the need for immunosuppressive drugs in cell transplantation. Additionally, the transparent, polymer microcontainer enables the non-invasive visualization of encapsulated cells using optical techniques and magnetic resonance imaging (MRI), which can help assess cell behavior, function and survival before and after grafting.

The microcontainer can be loaded with cells that secrete, for example, but not limited to, hormones, growth factors, inhibition factors, toxins, immune booster, or any combination thereof, or any molecule(s) that is/are directly therapeutic or elicits desired host response. In some embodiments, the present technology can be used to sequester cells for administration, or to deliver stem cells or other cells for regeneration. In a further embodiment, the microcontainers can be used sequester cells to localize them in the host and can have large pores to permit the vascularization of the encapsulated cells.

In a further embodiment, the microcontainer can also be loaded with drugs, pharmaceutical agents and imaging agents. The cells, drugs or agents can be released from the microcontainer to provide on-demand and localized delivery.

In some embodiments, the microcontainers can also be used for cell or bacteria encapsulation in applications where it is important to prevent the uncontrolled growth or proliferation of encapsulated cells or to prevent their migration into undesirable sites in the body. The microcontainers can also be used for the co-encapsulation of several cell types to permit cross-talk between the various cell types for strict control over levels of hormone and factors. In another aspect, this invention can be a nanocontainer used for the encapsulation of small biological material, drugs, pharmaceutical agents and contrast agents or a combination thereof.

The present technology provides method of making and use microcontainers. In some embodiments, the microcontainers can be polyhedral, spherical or other shapes, in one preferred embodiment, the microcontainers are cubes.

In an exemplary embodiment of the present technology, a novel biocompatible microcontainer can be fabricated comprising a hollowed cuboid base and an optically transparent nanoporous lid for containing one or more cells. A combination of nanoimprint lithography and oblique-angle metal deposition as well as conventional optical lithography may be utilized to make a dense array of narrow (down to ˜15 nm or less) nanoslots over large areas. Large areas include, but are not limited to, several square centimeters per imprint mold.

In some embodiments, the microcontainer is an epoxy-polymer-based or a highly cross-linked polymer based microcontainer, for example a photosensitive polymer, such as SU-8, may be utilized for the microcontainers that may serve as a molecular filter to immunoisolate cells in cell transplantation applications. The width of the nanoslots may be designed to allow bidirectional transfer of small molecules like oxygen and cellular nutrients, but prevent the entry of large molecules of the host immune system (e.g. immunoglobulins or complement factors), facilitating cell survival and function in an immunoisolated environment. In further embodiments, the microcontainer can be fabricated from, but not limited to, metal, glass, polymer, insulator, conductor or a combination thereof. Metallic containers can serve as electromagnetic shields; paramagnetic, ferromagnetic or diamagnetic containers can produce varying contrast in MRI. In any of these cases, the material surface can further be coated to enhance biocompatibility or biofriendliness, to prevent biofouling and biotoxicity, for example, but not limited to, can be coated with Polyethylene glycol (PEG).

The microcontainers of the present technology provide one or more of the following benefits for use in cell transplantation applications. First, the microcontainer material/surfaces is biocompatible and nonimmunogenic to either the graft tissue and the host tissue. Second, the microcontainer can encapsulate live cells without damage to the cells, such that the transplanted cells are protected from attack by the host immune system, specifically preventing the diffusion of large molecules such as immunoglobulins and complement proteins into the microcontainer. Third, the microcontainer allows the exchange of nutrients, cellular waste products, secretagogues, and hormones between the graft transplanted cells within the microcontainer and the host. Fourth, the microcontainer should be mechanically and chemically stable before and after transplantation. Fifth, the microcontainers provide adequate graft cell oxygenation and nutrients through the semi-permeable membrane (nanopores). Sixth, the microcontainers allow for implantation of cells to desirable locations, for example, well-vascularized or immunoprivileged sites without the risk of immune system attack. Lastly, the microcontainers can provide high-throughput, reproducible and cost effective manufacturing and biocompatibility of a system for use in cell therapy or transplantation.

In an exemplary embodiment of the present technology, a photosensitive polymer material, SU-8, manufactured by MicroChem Corp., Newton, Mass., may be utilized for the microcontainer. This material has demonstrated capability for MEMS drug delivery devices based on biocompatibility and biofouling characteristics. For example, chronic (51 weeks) recording of fiber spike signals using an SU-8-based neural probe implanted in 13 rats has been demonstrated without noticeable damage of tissue, which further supports the biocompatibility of SU-8-based cell transplantation devices. See Cho, S. Ju H, Cauller L, Romero-Ortega N, Lee J B, Hughes G. IEEE Sens J. 2008; 8:1830, incorporated by reference in its entirety.

The photoresist-based microcontainer 120 may be of cuboid shape and intended for cell transplantation, for example, islet transplantation. The microcontainer 120 may comprise a hollowed cuboid base 100 and a nanoporous lid 110 as shown in FIG. 1a). The container assembly can be achieved using, but not limited to, molecular bonding (molecular zippers/molecular velcro/etc.), magnetic assembly, chemical bonding or any combination thereof, or any other bonding mechanism including highly localized heat, temperature, pressure, or UV exposure. The nanoporous lid 110 may be assembled on top of the hollowed cuboid base 100 after the loading of cells inside the base 100. The devices can be loaded with cells via methods known in the art, including, but not limited to, manually loading or using a high-throughput manner with a microneedle array for example, and an array of thousands of devices can be loaded on a wafer, sealed with a wafer full of complementary lids, and then released in a biofriendly way by dissolving a sacrificial polymer in a biofriendly solvent.

Exemplary external dimensions of the hollowed cuboid base, include, but are not limited to, for example, about 300× about 300× about 250 μm³ or about 1100× about 1100× about 250 μm³. In some embodiments, box dimensions can be in the range from hundreds of microns down to sub-micron, depending on the biomolecule and/or end use application. In some embodiments, the thickness of the bottom face of the hollowed cuboid base 100 may be about 50 μm to about 450 μm and the width of the four side faces can be from about 50 μm to about 450 μm. In some embodiments, the small dimension microcontainer can be utilized for transplantation into a recipient and the large dimension microcontainer can be utilized for in vitro testing. In order to encapsulate live cells, the microcontainer inner space must be large enough to load at least one or more live cell and completely enclose the cells within its dimensions. In some embodiments, the inner dimensions of the microcontainer are 200 μm×200 μm×200 μm (8 nl), but in other embodiments, the inner dimensions may range in the hundreds of microns to submicron amounts depending on the biomolecule/s to be encapsulated and/or the end use application. The microcontainer of the present technology is semi-permeable and allow the free flow of nutrients, cellular waste products, and hormones, while restricting the entry of large molecules from the host immune system that are detrimental to cell survival. In some embodiments, the microcotainers of the present technology provide nanoslits of about 24.5 nm pores, which allows diffusion of insulin and glucose and can have about a 200× about 200× about 200 μm³ (8 nl) cubic cell encapsulation space.

In some embodiments, the nanoporous lid 110 may comprise an array of cylindrical wells, with about 30 μm diameter, for example, embedded into a 1100×1100×100 μm or 300×300×30 μm photoresist layer. The cylindrical well diameters may range from about 50 μm or less, and may be about 5 μm or less, and may include any range in between or below, or any range suitable to transport the desired molecules across the membrane but restrict unwanted molecules from access. The bottom surface of the nanoporous lid 110 may be sealed by a thin photoresist membrane, which can be, for example, about 350 to about 450 nm thick, for example, as illustrated in FIG. 1b). The thin photoresist membrane may comprise a dense array of about 25 nm wide slits, preferably about 20 nm wide slits, alternatively about 15 nm wide slits, alternatively about 10 nm wide slits, and includes any slit widths that would permit molecular transport. The thick photoresist slab in the nanoporous lid 110 may provide mechanical strength to the thin membrane whereas the thin membrane facilitates the rapid transport of nutrients and important cell signaling molecules. The nanoporous lid 110 may thus be able to withstand the pressures and thermal shocks applied during the fabrication process and also render size selective porosity to the device.

In some embodiments of the present technology, the microcontainers are used for transplant of cells into a recipient. In a preferred embodiment, the cells are islet cells. Transplanted islets have been used in insulin replacement therapy for type 1 diabetes which provides, as opposed to exogenous insulin sources, secreted insulin as a graded response to host glucose levels, more closely mimicking normal pancreatic function and can minimize many post-operative complications of organ (pancreas) transplantation. Islet also refers to Islet B cells that can sense an increase in blood glucose levels and secret insulin in response to it. In some embodiments, the islet cells can be allogeneic or xenogeneic. Islet xenotransplantation can be used which can overcome the severe shortage of human islets available for grafting. Effective immunoisolation of these xenografts can avoid a lifelong requirement of immunosuppressive drugs which has deleterious effects on beta cell function and on the host's ability to combat disease. Therefore, several researchers have focused on strategies to encapsulate islets so as to immunoisolate them for grafting.

The diameter of freshly isolated islets cells from a donor may be about 50 to about 300 μm range. Not to be bound by any particular theory, islets less than about 200 μm in diameter may result in better survival than large islets. In the present technology, both the small and large versions of microcontainers provide an encapsulation volume that may accommodate islets with a maximum diameter of about 200 μm.

In some embodiments of the present invention, the microcontainers are used in cell therapy or cell transplantation into a recipient. Cells may be obtained from any suitable donor, for example, any suitable mammal, including, a mouse, a dog, a pig, a rat, preferably a human. Recipients preferably include a mammal, for example, a mouse, a dog, a pig, a rat, preferably a human. In some embodiments, the transplantation is allogeneic, and in other embodiments, the transplantation can be xenogeneic.

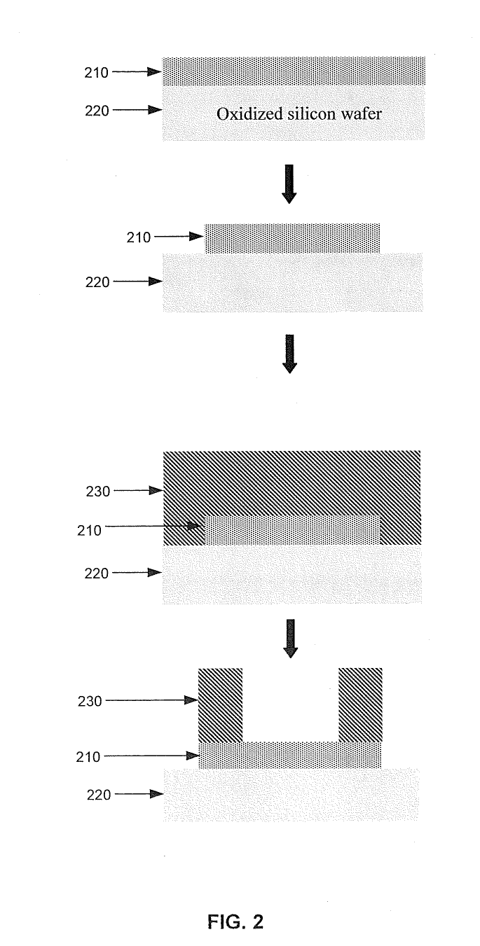

FIG. 2 is a diagram illustrating an exemplary process for fabricating a photoresist-based microcontainer, in accordance with an embodiment of the invention. Referring to FIG. 2, there is shown exemplary steps in the fabrication of the hollowed cuboid base 100. Steps a) through d) shows the process flow for the fabrication of the hollowed cuboid base 100. In step a), a 50 μm SU-8 2025 photoresist layer 210 may be spin cast on an oxidized silicon wafer 220 with a 2 μm oxide, for example. The photoresist layer 210 may be patterned using conventional optical lithography to form the bottom face of the hollowed cuboid base 100. In step b), a 200 μm thick SU-8 2075 photoresist layer 230 may be spun on the patterned 50 μm thick photoresist bottom face 210 and the oxidized silicon wafer 220, followed by a planarization process due to the high viscosity of the photoresist. The layers may then be baked and patterned using optical lithography to form the four side walls of the hollowed cuboid base 100. Finally, the hollowed cuboid bases may be released from the oxidized silicon wafer 220 by buffered oxide etchant (BOE).

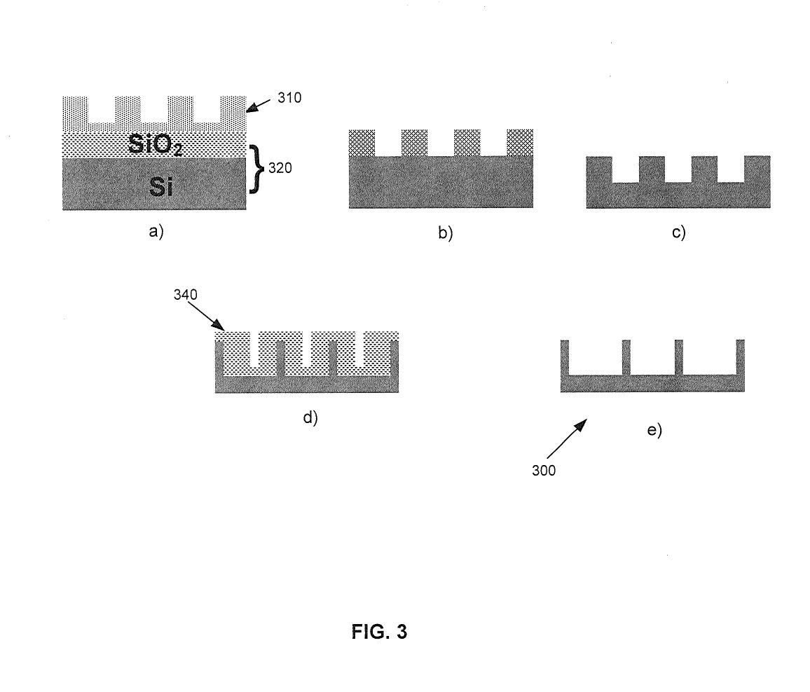

FIG. 3 is a diagram illustrating an exemplary process for fabricating a silicon mold for nanoimprinting, in accordance with an embodiment of the invention. Referring to FIG. 3, there is shown exemplary steps a) through e) for fabricating a silicon mold for nanoimprinting from an oxidized silicon wafer. In order to make 20 nm or smaller width nanoslots in a large area of photoresist such as SU-8, a silicon mold with approximately 20 nm width grating and 200 nm pitch may be fabricated for nanoimprinting.

To fabricate this mold, a layer of SU-8 (˜65 nm) may be spin coated on an oxidized silicon wafer 320 (˜50 nm SiO₂) and imprinted with a silicon master mold to yield a line and space grating over an area of ˜6 cm². The imprint with the master mold may be performed at 85° C. and 3 MPa for 15 minutes, resulting in the structure of FIG. 3a). Demolding may be performed at 35° C., followed by ultraviolet (UV) exposure with a dose of 450 mJ/cm² and post exposure bake at 95° C., for example. The imprinted SU-8 photoresist grating 310 may be transferred to the SiO₂ and then the silicon layer by a series of plasma etches in an inductively coupled plasma (ICP) etch system. In this process, the exposed SU-8 photoresist residue may be etched utilizing an oxygen plasma, followed by etching in a mixture of C₄F₈, CHF₃, and Ar to transfer the pattern down to the SiO₂ layer in the oxidized silicon wafer 320. Next, the pattern may be transferred into the Si layer by plasma etching in chlorine with exemplary etching conditions of 300 W ICP power, 100 W bias power, 5 mTorr, and 60° C. chuck temperature. This may result in an etch into the Si layer to a depth of ˜100 nm. An exemplary final pattern transferred to the Si layer in the oxidized silicon wafer 320 may comprise a line and space grating pattern with ˜140 nm Si lines separated by ˜60 nm spaces. After removal of the remaining SiO₂ mask, the resulting Si grating FIG. 3c), may be repeatedly oxidized in a furnace in O₂ at 900° C., oxidizing the surface of the silicon as shown in FIG. 3d), with the grown oxide 340 then etched by BOE to gradually reduce the grating dimension, resulting in the structure shown in FIG. 3e).

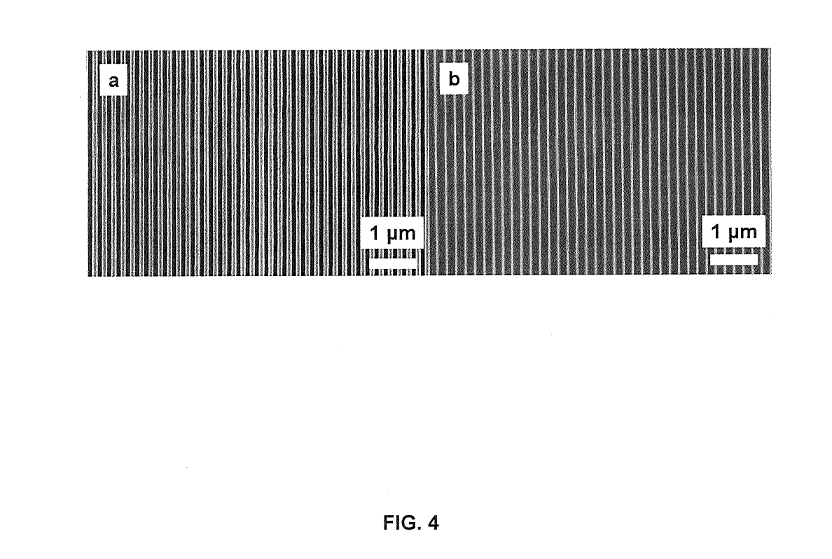

FIG. 4 illustrates top views from a scanning electron microscope of a silicon grating, in accordance with an embodiment of the invention. FIG. 4a) illustrates a top view scanning electron microscope (SEM) image of a silicon grating after the second oxidation and oxide removal step, while FIG. 4b) shows the final silicon mold following all oxidation and oxide etching steps, resulting in a structure with ˜20 nm wide silicon lines.

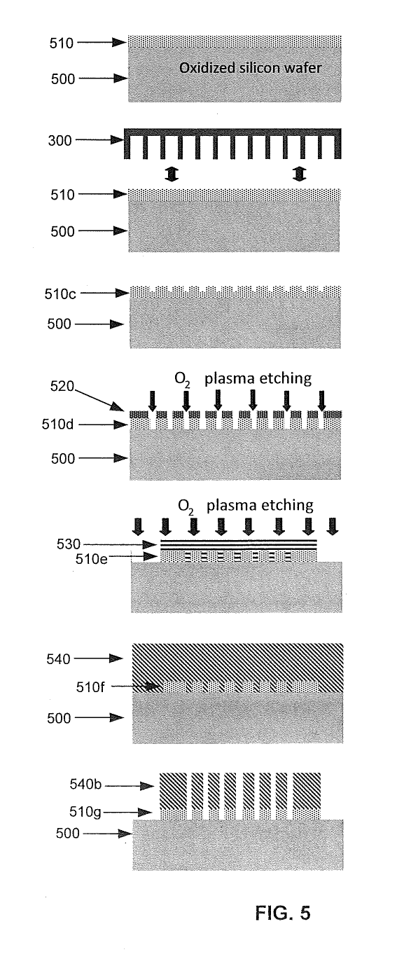

FIG. 5 is a diagram illustrating an exemplary process for fabricating a nanoporous lid for a microcontainer, in accordance with an embodiment of the invention. A layer of SU-8 photoresist 510 with a thickness of ˜450 nm may be spin coated on an oxidized Si wafer 500 with a ˜2 μm thick SiO₂ layer. The photoresist layer 510 may be imprinted with the 20 nm wide, 200 nm pitch nano-grating mold 300, fabricated as described with respect to FIG. 3, and imprinted with similar conditions, resulting in the imprinted photoresist gratings 510c. A chromium layer 520 may be selectively evaporated on the imprinted photoresist gratings 510c at 35°, followed by plasma etching in oxygen to etch exposed SU-8 photoresist, and Cr hard mask wet etching, resulting in the imprinted and etched photoresist layer 510d, as shown in FIG. 5d).

During plasma etching, there may be a slight widening of the nanoslots beyond the dimension of the top opening in the imprinted SU-8 photoresist layer 510d. However, due to the highly directional nature of ICP etching, the transferred dimension at the bottom of the trench is not significantly different from the topmost dimension. Also, as a result of the protection provided by the metal layer 520 on top of the imprinted SU-8 photoresist, the top dimension of the nanoslot remains unchanged. The dimension of the nanoslots may be scaled down further by evaporating metal at more oblique angles or by simply evaporating a thicker layer of metal which, in turn, reduces the gap.

After the formation of the nanoslots in the SU-8 photoresist membrane 510d, S1813 photoresist may be spin cast and patterned to form a 300 μm×300 μm or a 1100 μm×1100 μm square, as illustrated by the etched photoresist layer 510e in FIG. 5e). An O₂ plasma etch may be performed at 5 mTorr with an ICP RF power of 300 W and a bias RF power of 100 W, for example, to remove the 350-450 nm nano-slotted SU-8 membrane 510d except in the square area of 300 μm×300 μm or 1100 μm×1100 μm to create the footprint of the lid. After the nano-slotted SU-8 membrane patterning, the S1813 photoresist layer 530 may be removed with acetone, for example.

A 30 μm thick SU-8 2025 photoresist layer 540 may then be spin cast and patterned directly on top of the nano-slotted SU-8 membrane 510f, which resulted from the O₂ plasma etching shown in FIG. 5e), so that the nanoslots may be placed at the bottom of 30 μm diameter circular trenches, as shown in FIG. 5g). After the formation of a 30 μm thick SU-8 trench array 540b on top of the nano-slotted 350-450 nm SU-8 photoresist membrane 510g, nanoporous lids may be released from the oxidized silicon wafer 500 utilizing a BOE.

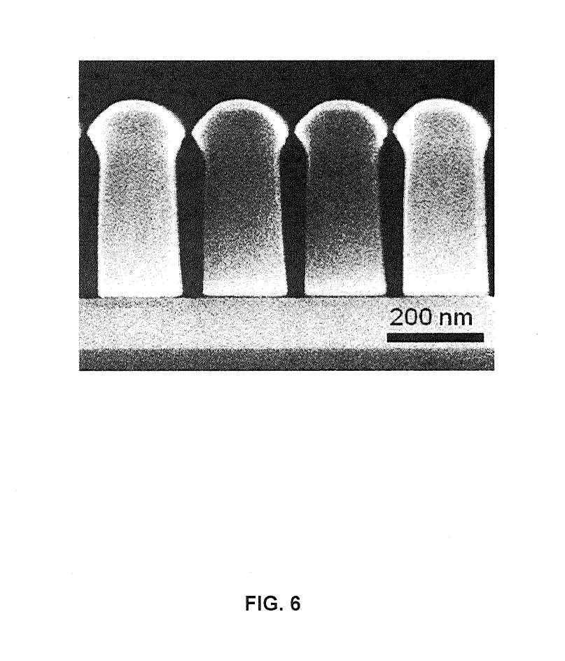

FIG. 6 shows a scanning electron microscope image of the cross section of an imprinted and etched photoresist membrane, in accordance with an embodiment of the invention. The openings may be 5-20 nm wide at the top.

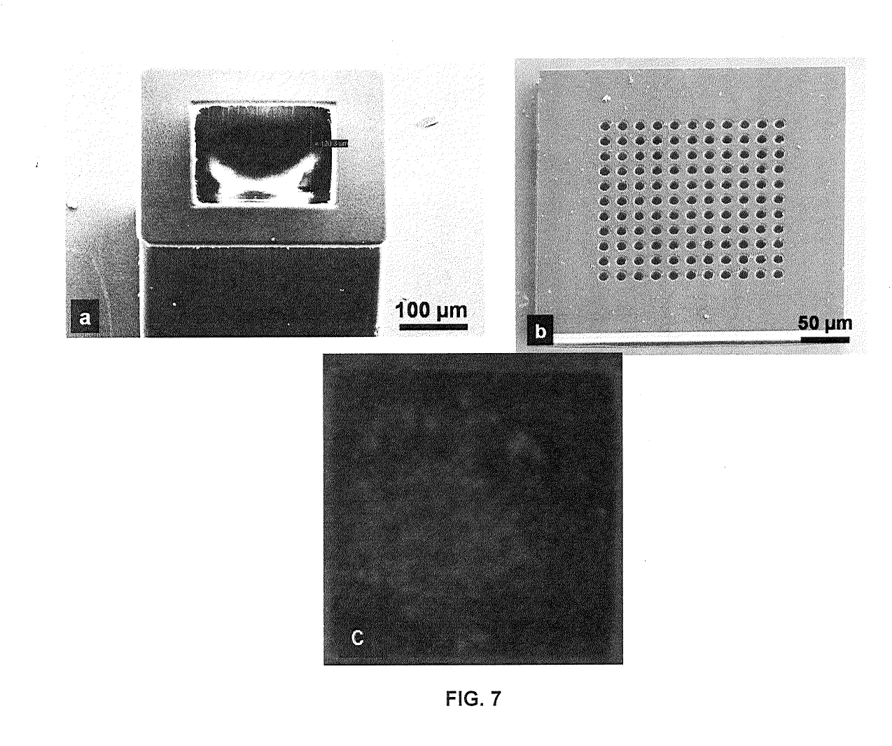

FIG. 7 shows scanning electron microscope images of a microcontainer with nanoporous lid, in accordance with an embodiment of the invention. Referring to FIGS. 7a and 7b, there is shown an exemplary 300×300×250 μm hollowed cuboid base and a 300×300 μm nanoporous lid. For a 1100 μm×1100 μm nanoporous lid, a 100 μm thick SU-8 photoresist may be used instead of 30 μm SU-8 2025 photoresist.

Islets were maintained in modified RPMI medium supplemented with 10% FBS, 1% Penicillin/Streptomycin and 2% INS-1 solution (0.5 M HEPES sodium salt; L-Glutamine, 100 mM; Sodium pyruvate, 50 mM; B-mercaptoethanol, 2.5 mM; pH 7.4). Islets were pipetted into the encapsulation space of the cuboid base and allowed to settle under gravity. The bases were then closed with the nanoporous lids and maintained in a tissue culture dish.

The size-dependant selective molecular porosity of the nanoporous microcontainer was verified using the islet-specific fluorescent probes lectin-FITC (140 kDa) and FM 4-64 (608 Da). The lectin conjugate was selected because it is slightly smaller than the immunoglobulins and complement proteins of the host immune system, while FM 4-64 molecule was selected because it is larger than the cell signaling molecules like insulin and glucose. Lectin-FITC staining solution was prepared by adding 120 μl of lectin-FITC (140 kDa; 1 mg/ml in PBS; www.sigma.com) to 720 μl of RPMI medium. FM 4-64 (607 Da; www.sigma.com) solution was prepared in HBSS at a concentration of 1 μg/ml.

200 μl of lectin-FITC solution was added to the tissue culture dish containing the microcontainers and the dish was incubated at 37° C., 5% CO2 for 24 h. At the end of the 24 h, 2 μl of FM 4-64 solution was added followed by an additional 30 min incubation. The medium was then removed and the microcontainers were washed thrice with PBS to remove the unbound dye. The microcontainers were then observed with a Leica TCS SP5 laser scanning confocal microscope. Both dyes were excited at 488 nm, using the microscope's argon ion laser. The lectin-FITC fluorescence was observed in the green channel (530 nm) and the FM 4-64 was observed in the red channel (630 nm). Images were processed using ImageJ 1.42 software as shown in 7c.

Our fabrication scheme resulted in the successful creation of small microcontainers for transplantation applications. Larger microcontainers, with the same encapsulation volume and surface nanoporosity, were similarly created for in vitro testing. The thick walls of the microcontainers ensured that they never ruptured during fabrication and subsequent experiments. The hollowed cuboid housed islets without entrapping them, which is a more physiological approach to grafting as compared with alginate microbeads that entrap and immobilize cells for encapsulation. The use of SU-8 rendered the microcontainers transparent to light and radio frequency waves, which is critical in studying the post encapsulation behaviour of cells using optical techniques and magnetic resonance imaging, respectively. Additionally, SU-8 can be easily modified to modulate its porosity or functionalized with biosensors that can report on the in vivo microenvironment of the grafts. See, for example, C-J Chang, C-S Yang, Y-J Chuang, H-S Khoo and F-G Tseng, Nanotechnol. 19 1 (2008), incorporated by reference in its entirety.

The cyclic oxidation and etching process resulted in an imprint mold with the desired grating width. The mold had the mechanical strength necessary for repeated imprinting. A robust mold for repeated and reproducible imprinting is critical for the high throughput creation of nanoporous membranes in applications such as islet transplantation in diabetics because the process requires the grafting of hundreds of thousands of islets to restore glycemic control in the patient.

Nanoimprinting in SU-8 resulted in the desired nanoslot width at the top of the nanoslot cross-section. The nanoslot cross-section was narrowed at the bottom after etching, thereby creating additional impedance to the transport of large molecules. The imprinting was carefully performed so that there was no flexing of the SU-8 membrane that could result in nanoslot homogeneity across the membrane.

Islet survival in SU-8 nanoporous microcontainers was tested to confirm biocompatibility. See, B. Gimi, J. Kwon, A. Kuznetsov, B. Vachha, R. L. Magin, L. H. Philipson and J. B. Lee, J Diabetes Sci Technol 3, 297 (2009), incorporated by reference in its entirety. In this study, islets within the microcontainers were incubated in the presence of large and small molecules to ascertain the microcontainer's porosity and impedance to molecular transport. Confocal imaging showed the penetration of the small molecule dye FM 4-64 into the microcontainer, as shown in FIG. 7c). This result is encouraging because FM 4-64 is larger than insulin and glucose, suggesting the exchange of nutrients, growth factors secretagogues and hormones necessary for graft survival and function. We also observed some penetration of the large molecule dye, which suggests that some large molecules of the immune system may penetrate into the microcontainer since the lectin is slightly larger than the smallest immunoglobulin. However, the mere presence of molecules is not harmful to the graft; a key factor in graft survival is whether complement molecules are active when they arrive at the graft.

While the invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiments disclosed, but that the present invention will include all embodiments falling within the scope of the appended claims.

What is claimed is:

1. A method of immunoisolating one or more biomolecules, the method comprising:
providing a biocompatible photoresist-based microcontainer, wherein the photoresist-based microcontainer comprises a base and semi-permeable nanoporous surface, wherein the nanoporous surface comprises a thin nanoporous membrane comprising a dense array of uniform slits and a thick photoresist support layer comprising a uniform array of cylindrical wells; and
encapsulating the one or more biomolecules within the biocompatible microcontainer.

2. Wherein the at least one or more biomolecules is at least one or more cells, one or more agents or one or more pharmaceutical drugs.

3. The method of claim 1, wherein the base is fabricated utilizing optical lithography of a photoresist layer on an oxidized silicon substrate, and
wherein the nanoporous membrane is fabricated utilizing nanoimprinting of a photoresist layer on an oxidized silicon substrate

4. The method according to claim 1, wherein the cylindrical wells have a diameter of about 30 μm or less and wherein the slits are less than 25 nm wide.

5. The method according to claim 4, wherein the slits are less than about 20 nm wide.

6. The method according to claim 4, wherein the slits are less than about 15 nm wide.

7. The method according to claim 1, wherein the biocompatible photoresist-based microcontainer comprises SU-8.

8. The method according to claim 2, wherein the one or more cells are islet cells.

9. The method according to claim 8, wherein islet cells are allogeneic or xenogeneic cells.

10. The method according to claim 1, wherein the nanoporous membrane is permissive to the passage of one or more molecules.

11. The method according to claim 10, wherein the one or more molecule is selected from the group consisting of hormones, growth factors, inhibition factors, toxins, immune booster, small biological material, drugs, pharmaceutical agents, and any combination thereof.

12. A method of treating a disease in a patient, comprising administering one or more biomolecules which have been immunoisolated by the method of claim 1 to the patient.

13. The method of claim 12, wherein the one or more biomolecules comprise one or more cells, one or more pharmaceutical agents or one or more drugs.

14. The method of claim 12, wherein the disease is selected from the group consisting of hormone deficiency diseases, diabetes, parathyroid disease, Parkinson's disease, hemophilia, Alzheimer's disease, CNS malignancies, hypothyroidism, and cancer.

15. The method of claim 13, wherein the disease is diabetes and the one or more cells comprise islet cells.

16. The method of claim 15, where the biocompatible photoresist-based microcontainer allow passage of insulin through the biocompatible photoresist-based microcontainer.

17. A method of treating a disease or disorder in a patient comprising:
administering to the patient one or more biomolecules encapsulated within an immunoisolating photoresist-based microcontainer,
wherein the immunoisolating photoresist-based microcontainer comprises a base and semi-permeable nanoporous surface, wherein the semi-permeable nanoporous surface comprising a thin nanoporous photoresist membrane comprising a dense array of slits and a thick photoresist support layer comprising an array of cylindrical wells; and
wherein the immunoisolating photoresist-based microcontainer comprises a biocompatible and non-immunogenic composition.

18. The method according to claim 17, wherein the disease is selected from the group consisting of hormone deficiency diseases, diabetes, parathyroid disease, Parkinson's disease, hemophilia, Alzheimer's disease, CNS malignancies, hypothyroidism, and cancer.

19. The method according to claim 18, wherein the disease or disorder being treated is diabetes and wherein the one or more cells are islet cells.

20. A system for transplantation, the system comprising:
a photoresist-based microcontainer for biotherapeutic molecule transplantation, wherein said microcontainer comprises a cuboid base and a nanoporous lid, said nanoporous lid comprising a thin nanoporous photoresist membrane and a thick photoresist support layer.

21. The system according to claim 20, wherein said cuboid base is fabricated utilizing optical lithography of a photoresist layer on an oxidized silicon substrate.

22. The system according to claim 20, wherein said nanoporous membrane is fabricated utilizing nanoimprinting of a photoresist layer on an oxidized silicon substrate.

23. The system according to claim 22, wherein said nanoimprinting comprises imprinting a pattern into a photoresist layer utilizing a silicon nanograting mold.

24. The system according to claim 22, wherein said nanoimprinting comprises oblique-angle deposition of metal onto said imprinted pattern in said photoresist layer.

25. The system according to claim 22, wherein said thin nanoporous photoresist membrane is fabricated in a bottom layer of said nanoporous lid.
